(19) United States
(12) Patent Application Publication
(10) Pub. No.: US 2016/0238583 A1
(43) Pub. Date: Aug. 18, 2016

(54) SENSOR FOR METAL DETECTION

(71) Applicant: KING ABDULLAH UNIVERSITY OF SCIENCE AND TECHNOLOGY, Thuwal (SA)

(72) Inventors: Rimantas KODZIUS, Thuwal (SA); Guoqing ZHAO, Hong Kong (CN)

(73) Assignee: KING ABDULLAH UNIVERSITY OF SCIENCE AND TECHNOLOGY, Thuwal (SA)

(21) Appl. No.: 14/654,506

(22) PCT Filed: Dec. 20, 2013
(86) PCT No.: PCT/IB2013/003241

§ 371 Date: Jun. 19, 2015

Related U.S. Application Data
(60) Provisional application No. 61/740,271, filed on Dec. 20, 2012.

(51) Int. Cl. G01N 33/20; G01N 33/84; G01N 27/48; B01L 3/00

(52) U.S. Cl. CPC G01N 33/20; B01L 3/502715; B01L 3/50273; G01N 33/84; G01N 27/48; B01L 2300/0645; B01L 2300/18; B01L 2300/0627; B01L 2300/14; B01L 2300/0883; B01L 2200/0605

(57) ABSTRACT

A sensor for monitoring and detecting metals in a sample is provided. Methods and systems for monitoring and detecting metals in a sample are also provided.

### CLAIM OF PRIORITY

This application claims the benefit of prior U.S. Provisional Patent Application No. 61/740,271, filed on Dec. 20, 2012, which is incorporated by reference in its entirety.

### TECHNICAL FIELD

This invention relates to a sensor for metal detection, including toxic metals, and methods and systems relating to the sensor.

### BACKGROUND

The current standard techniques for trace metal analysis include Inductively Coupled Plasma-Mass Spectrometry (ICP-MS), Inductively Coupled Plasma Optical Emission Spectrometer (ICP-OES), and Atomic Absorption Spectrometry (AAS). These methods require bulky and expensive equipment, which cannot be used in the field. Moreover, gaseous effluents are produced from the above-mentioned methods that can be difficult to treat or dispose of. Additionally, such methods require complicated and time-consuming sample pre-concentration and treatment steps to be carried out by trained professionals. As such, there is currently a need for rapid detection and measurement of metals in field samples using portable analytical instruments.

### SUMMARY

Anodic Stripping Voltammetry (ASV) or Adsorptive Stripping Voltammetry (AdSV) are techniques for qualitative and quantitative analysis. These techniques are currently a versatile solution for on-site detection of metals, e.g., heavy metals. Previous approaches of heavy metal detection using ASV/AdSV methods involve labor intensive work. In ASV/AdSV methods every step of detection needs be operated manually and the detection requires high volume sample and reagent. The total size and mass of the instrument and all of the reagents necessary for operation is not easily portable and it is difficult to use for on-site operation.

In one aspect, a sensor for detecting a metal in a sample includes a microfluidic flow channel including an inlet port, an outlet port, and a detection chamber including a group of sensing electrodes including a working electrode, a counter electrode, and a reference electrode; a flow sensor configured to measure flow in the channel; a temperature sensor configured to measure temperature in the channel; and an electrical connection configured to connect the sensor to a sensing device.

The group of sensing electrodes can include two interdigitated electrodes and one serpentine electrode arranged between the interdigitated electrodes. The sensor can further include a micro-heater configured to heat a sample in the flow channel. The sensor can further include a pH sensor configured to measure a pH of a sample in the flow channel. The sensor can further include one or more sample filters.

The sensor can be configured to selectively detect one or more metals selected from chromium (Cr), manganese (Mn), iron (Fe), cobalt (Co), nickel (Ni), copper (Cu), zinc (Zn), arsenic (As), selenium (Se), silver (Ag), cadmium (Cd), tin (Sn), antimony (Sb), tellurium (Te), gold (Au), mercury (Hg), titanium (Ti), lead (Pb), bismuth (Bi), and a combination thereof.

The flow sensor can be a thermal differential sensor. The sensor can be arranged on a glass substrate. The sensor can further include a reagent chamber configured to deliver a reagent to the flow channel. The reagent can be a standard solution of copper (Cu), lead (Pb), cadmium (Cd), or a combination thereof. The electrodes can be composed of a non-toxic material. The non-toxic material can include silver (Ag), gold (Au), platinum (Pt), bismuth (Bi), graphite, or glassy carbon. The electrodes can be composed of mercury (Hg).

In another aspect, a system for detecting a metal in a sample includes a sensing device; and a sensor including: a microfluidic flow channel including an inlet port, an outlet port, and a detection chamber including a group of sensing electrodes including a working electrode, a counter electrode, and a reference electrode; a flow sensor configured to measure flow in the channel; a temperature sensor configured to measure temperature in the channel; and an electrical connection configured to connect the sensor to the sensing device.

The sensing device can be further connected to a computer system. The computer system can be a smartphone. The computer system further includes a computer-readable storage medium having computer-readable program code stored therein, the computer-readable program code including instructions for controlling a detection process; analysis of detection result data; and/or visualization of detection result data.

In another aspect, a method of using a sensor for detecting a metal in a sample includes providing a sensor including: a microfluidic flow channel including an inlet port, an outlet port, and a detection chamber including a group of sensing electrodes including a working electrode, a counter electrode, and a reference electrode; a flow sensor configured to measure flow in the channel; a temperature sensor configured to measure temperature in the channel; and an electrical connection configured to connect the sensor to a sensing device; introducing a sample to the flow channel via the inlet port; allowing the sample to flow to the detection chamber; and detecting a metal in the sample using the group of sensing electrodes.

Allowing the sample to flow can include applying negative pressure to the outlet port. The pressure can be selected to maintain a constant flow rate in the range of 0.1 ml/min to 100 ml/min. Allowing the sample to flow can include using capillary action. Allowing the sample to flow can include applying positive pressure to the inlet port.

The method can further include measuring a flow rate or a flow volume of the sample in the flow channel. Measuring the flow rate or the flow volume can include using a thermal differential sensor. The method can further include measuring a temperature of the sample in the flow channel.

The method can further include applying a deposit potential between the working electrode and the counting electrode for a period of time. The method can further include applying a hold potential between the working electrode and the counting electrode for a period of time. The method can further include applying a strip potential between the working electrode and the counting electrode for a period of time. The method can further include measuring a current which flows through the counting electrode using a sensing device.

A current peak can be obtained from the measured current and compared with a standard measurement to determine the type of metal detected and/or the concentration of metal in the sample. Detecting a metal using the group of sensing electrodes can include ASV or AdSV.

The sample can be a clinical sample, water sample, food sample, air sample, or soil sample. The food sample can include a liquid. The clinical sample can include stool, saliva, sputum, bronchial lavage, urine, vaginal swab, nasal swab, biopsy, tissue, tears, breath, blood, serum, plasma, cerebrospinal fluid, peritoneal fluid, pleural fluid, pericardial fluid, joint fluid, or amniotic fluid.

The details of one or more embodiments are set forth in the drawings and description below. Other features, objects, and advantages will be apparent from the description, the drawings, and from the claims.

### BRIEF DESCRIPTION OF THE DRAWINGS

FIG. 1 is a graphic depicting a sensor prototype.

FIG. 2 is a comparison of a secure digital (SD) card and two sensors.

FIG. 3 is a graph depicting operating configuration of a sensor.

FIG. 4 is a graphic depicting a sensing device.

FIGS. 5a-5g depict different electrode configurations.

### DETAILED DESCRIPTION

The sensor described herein provides an excellent solution for on-site metal detection, including heavy metal detection. Compared with conventional ASV and AdSV heavy metal detection methods, the sensors described herein provide significant advantages in higher throughput, lower cost, at the same time being less labor intensive and less dependent on individual skills. Additional benefits include the disposable design of the sensor, the enhanced reliability and repeatability of measurements. The sensors can be widely applied in various industries such as but not limited to clinical diagnostics (biopsy tests, excretory tests—using saliva, blood, blood plasma or serum, feces, urine, tears, sweat, etc. as samples), environmental protection, food industry, agriculture and veterinary settings. A device comprising the sensors can be used not only in an industrial or environmental setting, but also in, e.g., a doctor's office, or a home setting.

The sensor for metal detection as described herein is based on microfluidic technology. A sensor for use with a portable analytical instrument is configured for detection of metals (such as toxic metals) in solids (food, soil, etc.), liquids (water, juices and other drinks, clinical samples such as blood samples, waste samples, bodily fluid samples, etc.) and gases (air, etc.). The sensor can be supplied with pre-stored chemical reagents as desired, and can be used with complementary analytical software.

The detection of metals is based on ASV or AdSV. The detectable metals may vary depending on different chip designs which may use different electrode configurations. The sensor can be used to detect metals, including but not limited to metal ions, metal complexes and metal compounds. Metals that can be detected include but are not limited to chromium (Cr), manganese (Mn), iron (Fe), cobalt (Co), nickel (Ni), copper (Cu), zinc (Zn), arsenic (As), selenium (Se), silver (Ag), cadmium (Cd), tin (Sn), antimony (Sb), tellurium (Te), gold (Au), mercury (Hg), titanium (Ti), lead (Pb) or bismuth (Bi). As an example, sensors with mercury (Hg) working electrodes can be used to detect metals that include but are not limited to Zn, Fe, Pb, Cu, Bi, Cd, etc. In another example, sensors with carbon (graphite or glassy carbon) working electrodes can be used to detect metals that include but are not limited to Hg, Ni, Co, Cr, Au, Fe, etc. In another example, sensors with bismuth (Bi) working electrodes can be used to detect metals that include but are not limited to Cd, Pb, Cu, Ti, Zn, Ni, Co, Cr, etc. In another example, sensors with gold (Au) working electrodes can be used to detect metals that include but are not limited to As and Hg.

Sensor

In general, a sensor can include one or more electrodes positioned on a substrate. The substrate can be composed of one more materials. Suitable substrate materials include, for example, glass, silicon, a ceramic, plastic, wax, paper, or other material that can support the electrode(s).

Different sensors can be used for blank data measurement, standard curve measurement, sample estimation and sample measurement based on the desired chip function. For example, a calibration sensor chip is a sensor chip that contains pre-calibration data. It can be used to upload the calibration data of one pack of sensors to a sensor device and it also can be used to measure a standard sample solution to perform on-site calibration.

A sample estimation sensor chip can be used to detect sample composition and concentration of a field sample. It can also be used to select optimized parameters for a measurement. The sample estimation chip may include one or more sets of sensing electrodes which can be used for many measurements without replacing chip. It also may include a pH sensor, for example, an ion sensitive field effect transistor (ISFET).

In addition, based on different detectable metals, there can be various types of the sensor. For example, different sensors can be used for detection of different kinds of metals. Alternatively, one sensor can be used for detection of several kinds of metals.

The sensor for metal detection can include an inlet port for sample injection, an outlet port for sample extraction, a channel, and two or more electrodes. The electrodes can include processing and sensing electrodes, a temperature sensor and one or more electrodes to connect the sensor to a sensing device. The sensor optionally further comprises one or more of a flow sensor, a temperature sensor, a pH sensor, and one or more reagents. For example, the sensor can include a flow sensor to measure liquid flow volume and flow rate. Volume and flow rate can be important parameters for quantitative measurement and analysis. In particular sample volume can be important for metal concentration calculation, and a constant flow rate can be important for metal deposition. Steady flow (e.g., static flow or a constant rate of flow) of sample fluid can be important during measurement. In another embodiment, the sensor can include a temperature sensor to measure the sample temperature.

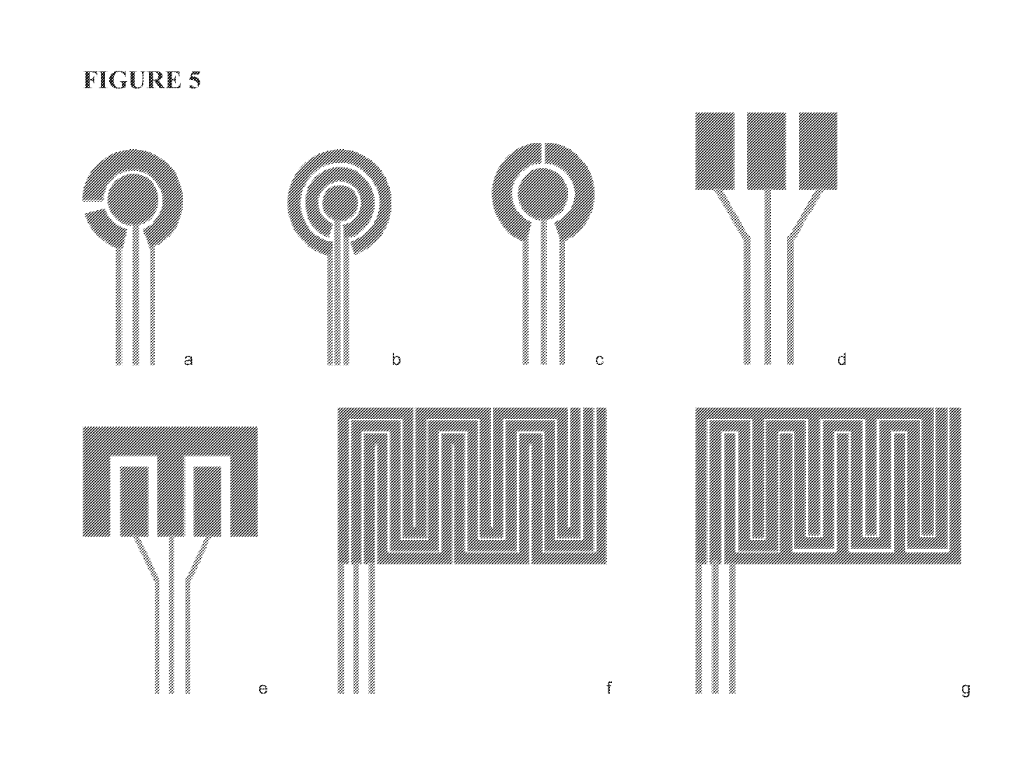

The working, counter, and reference electrodes can be formed in a variety of configurations. Some exemplary configurations are illustrated in FIGS. 5a-5g. For example, FIG. 5d illustrates the working, counter, and reference electrodes as three parallel electrodes. FIG. 5g illustrates the working, counter, and reference electrodes as two interdigitated electrodes with a serpentine electrode arranged between the interdigitated electrodes. The configuration of the working, counter, and reference electrodes can be selected so as to provide high surface area on a single surface while minimizing the distance between the electrodes. Such a design helps increase sensor performance and keep the cost of the sensor low.

The sensing electrodes in a sensor can be used to detect metal ions in a sample. In one embodiment, reagents can be pre-stored on chip or are provided to the chip just before detection. In one embodiment, pre-processing steps can include sample filtering, conductivity enhancing for field samples or sample pre-concentration. Field samples can be more complex than samples prepared in the laboratory. Without these pre-processing steps, ASV may not work for field samples such as pipe water, drinking water, juice, etc. For example, the conductivity of pipe water or drinking water samples may be too low to perform the detection of heavy metals and the particles within those samples may contaminate the sensor electrodes and block the channels of the sensor. Suitable reagents for sample pre-processing for example, sample digestion or enhancement of sample conductivity and for sensing electrodes processing such as mercury (Hg) thin film electroplating can be used. For some specific samples, the reagents can be used to react with the sample for detection. For example, a standard solution (e.g. a solution of KNO₃ and HNO₃) can be used as a supporting electrolyte. A supporting electrolyte is sometimes desirable for analysis of low conductivity samples, such as clean drinking water. This solution can be mixed with the sample before detection. Mixing can be performed in a sample vessel or on the sensor chip using an on-chip microfluidic mixer.

In one embodiment, processing electrodes can be used to enhance the reaction of the sample with reagents. For example, a micro-heater can be used to heat up the mixture of sample and reagents to enhance sample processing. Any micro-heater suitable for use within a sensor, e.g. a platinum micro-heater, can be used. The sensor can include at least one inlet and one outlet for sample deposit and extraction. The sample can be injected into the sensor via the inlet. Capillary force, negative pressure force or positive pressure force can be used to manipulate sample and reagent flow on a sensor. For example, a peristaltic pump, vacuum source, or other apparatus that can apply negative pressure, may be used to extract air from a waste fluid vessel to keep a constant negative pressure. This negative pressure can be used to draw fluid from the outlet and into the waste vessel. The sensor can also include one or more filters for sample filtering and pre-concentration. The sensor can include a flow channel through which liquid sample and optional reagent flow.

Alternatively, the sensor can be a probe sensor chip without a flow channel. A probe sensor chip lacks a cover, which in other embodiments forms fluid channels. The probe sensor chip can simply be dipped into a sample for measurement.

In another embodiment, a calibration chip for measuring a reference sample and recordation of data as reference for the measurement of a batch of sensors is included. For quantitative analysis, the ASV method requires a standard sample measurement for comparison calculation.

Device

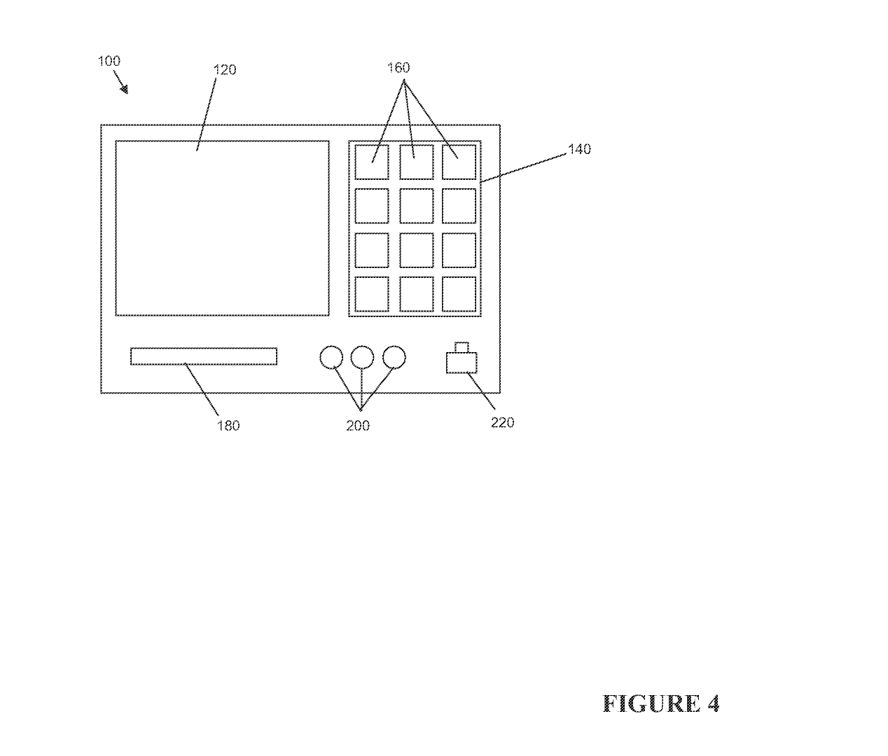

Referring to FIG. 4, the sensor can be connected to a sensing device 100 through connection port 180. Connecting electrodes within connection port 180 serve to electrically connect the device to the sensor. The device can be a hand-held or portable device. The device can optionally be connected to a computing system. The computing system can include a computer, a mobile phone, a smartphone or any other suitable computing system. The device can control sample deposit, sample pre-processing, electrode pre-processing, reaction of sample with reagents, signal sensing and data processing. The device can provide a desired potential between the working electrode, the reference electrode, and the counting electrode on the sensor. The device can measure electrical properties at the electrodes, e.g., the current at the counting electrode. The device can receive input from the sensor, e.g., from the flow sensor, temperature sensor, or other systems on the sensor.

The device can be configured to control peripheral components, e.g., a source of negative pressure which is connected to the outlet. In this way, the device can provide feedback, adjusting the negative pressure in response to changes in flow rate, so as to provide a stable flow rate through the flow channel.

Software can be included to assist with the detection process control, result data analysis and visualization. The software may be embedded into a device or run on a computer, mobile phone or other computing system.

In one embodiment, a device 100 can include a display 120 and an input region 140. The device 120 can be used to display images in various formats, for example, joint photographic experts group (JPEG) format, tagged image file format (TIFF), graphics interchange format (GIF), or bitmap. The display 120 can be used to display text messages, help messages, instructions, queries, test results, and various information to the users. In some implementations, the display 120 can support the hypertext markup language (HTML) format such that displayed text may include hyperlinks to additional information, images, or formatted text. The display 120 can further provide a mechanism for displaying videos stored, for example in the moving picture experts group (MPEG) format, Apple's QuickTime format, or DVD format. The display 120 can additionally include an audio source (e.g., a speaker) to produce audible instructions, sounds, music, and the like. The input region 140 can include keys 160 or can be implemented as symbols displayed on the display 120, for example, a touch sensitive screen. The device 120 can further include a communication port 220. A communication port 220 can be, for example, a connection to a telephone line or computer network.

In another embodiment, the device 100 can access programs and/or data stored on a storage medium (e.g., video cassette recorder (VCR) tape or digital video disc (DVD); compact disc (CD); floppy disk; flash drive; hard disk; or a cloud system). Additionally, various implementations may access programs and/or data accessed stored on another computer system through a communication medium including a direct cable connection, a computer network, a wireless network, a satellite network, or the like.

A device may be implemented using a hardware configuration including a processor, one or more input devices, one or more output devices, a computer-readable medium, and a computer memory device. The processor may be implemented using any computer processing device, such as, a general-purpose microprocessor or an application-specific integrated circuit (ASIC). The processor can be integrated with input/output (I/O) devices to provide a mechanism to receive sensor data and/or input data and to provide a mechanism to display or otherwise output queries and results to a service technician. Input devices include, for example, one or more of the following: a mouse, a keyboard, a touch-screen display, a button, a sensor, and a counter.

The display 120 may be implemented using any output technology, including a liquid crystal display (LCD), a television, a printer, and a light emitting diode (LED). The computer-readable medium provides a mechanism for storing programs and data either on a fixed or removable medium. The computer-readable medium may be implemented using a conventional computer hard drive, or other removable medium such as those described above with reference to. Finally, the system uses a computer memory device, such as a random access memory (RAM), to assist in operating the sensor device.

The device can provide access to applications such as a toxic metals database or other systems used in monitoring toxic metals. In one example, the device connects to a toxic metal database via communication port. The device may also have the ability to go online, integrating existing databases and linking other websites. Online access may also provide remote, online access by users to toxic metals detection, levels and treatment. The device can be used in an industrial setting, an environmental setting, or any desired location.

Also provided is a system for detecting toxic metals which can include a portable instrument or device and interchangeable sensors based on microfluidic technology.

Kit

Further provided is a kit for detecting metals that can be used with a portable instrument or device as depicted in FIG. 4 for example. The kit can include instructions for taking a sample and/or for detecting or measuring toxic metals, and one or more sensors for detecting toxic metals. The sensors can be reusable or disposable. The kit can further comprise reagents for detecting toxic metals or for use as a standard. The instructions for taking a sample and/or for detecting or measuring toxic metals may be optional. A device can be included in the kit as well. Such a device can be a portable or a handheld device that measures or detects the presence of toxic metals, allows manual or automatic input of the results, allows the identification of the metals detected or allows the evaluation of the levels of the metals detected.

### EXAMPLES

Several chips were designed, fabricated and tested in laboratory. The electrodes were fabricated on a piece of soda-lime glass substrate with micro-fabrication processes, e.g., sputtering, electro-beam evaporation, lift-off, and so on. The channels were fabricated using PDMS (polydimethylsiloxane) material with molding technology. Then the channel layer and the substrate were aligned and adhered together to form the sensors. Ceramic, glass, polymer, or other substrates can be used with modification of the fabrication process.

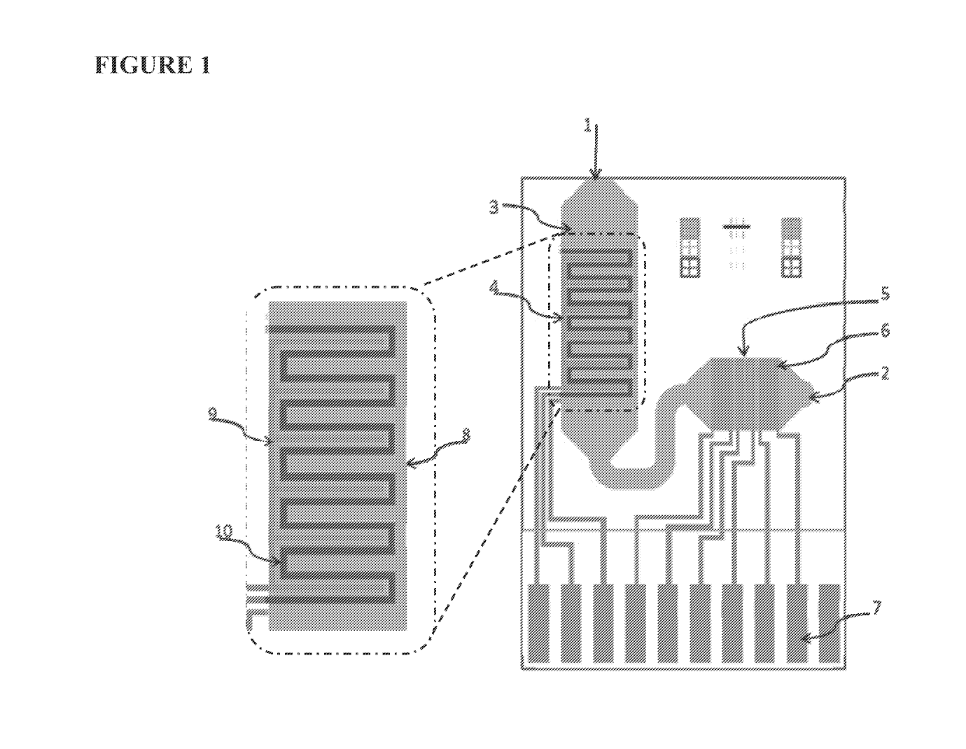

Referring to FIG. 1, a sample inlet 1 guides a sample into the device. Sample outlet 2 guides the sample out of the device. A negative pressure pump may be connected to outlet 2. Channel 3 guides sample flow through a detection chamber and a flow rate/temperature sensing chamber. The channel 3 can be formed between the chip substrate and its cover, which can be fabricated from PDMS. Sensing electrodes 4 detect metals. The sensing electrodes 4 include working electrode 8, counting electrode 9, and reference electrode 10. Thermal differential sensor 5 measures sample flow rate and flow volume. Temperature sensor 6 measures sample temperature. Connecting electrodes 7 are used to connect the sensor to the instrument.

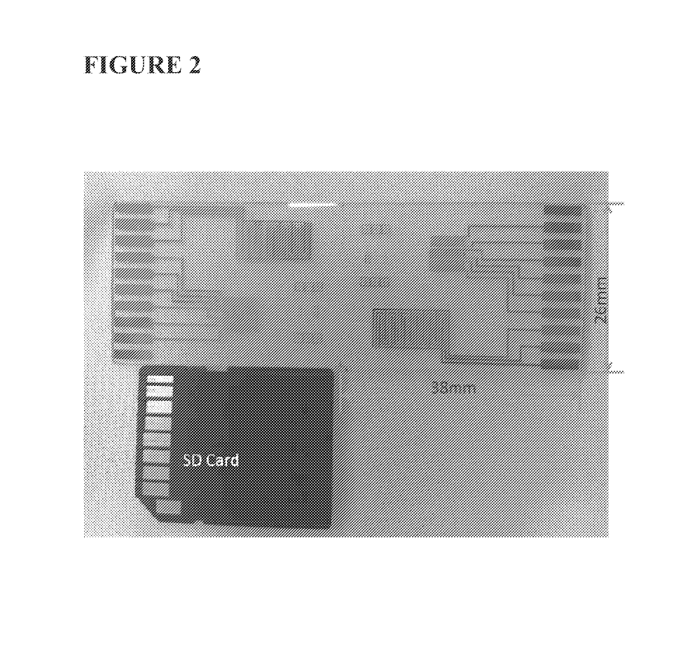

As shown in FIG. 2, the prototype chips were fabricated on a glass substrate. The electrodes were formed by using sputtering and e-beam evaporation processes. Micro/nano fabrication technologies were employed in the fabrication process. The device cover (not shown) was fabricated using PDMS material.

Operating Process and Operating Parameters

The operating parameters of the sensor which used a Hg film as working electrode are listed in Table 1. These parameters were chosen for measurement of Cu, Pb and Cd in a sample in a concentration range of 10 ppb to 100 ppb (parts-per-billion).

For one packaged sensor, the standard reference data can be calibrated by the manufacturer and recorded on the calibration sensors. The standard reference data can also be modified by a user by performing a measurement of a standard solution with calibration sensors.

Test experiments were performed using one sensor to measure a standard solution for reference and calculation. Then after a cleaning operation, the chip was used to measure a sample solution. The cleaning operation uses a positive potential to strip metal from the working electrode completely and return working electrode to its original state before a further measurement.

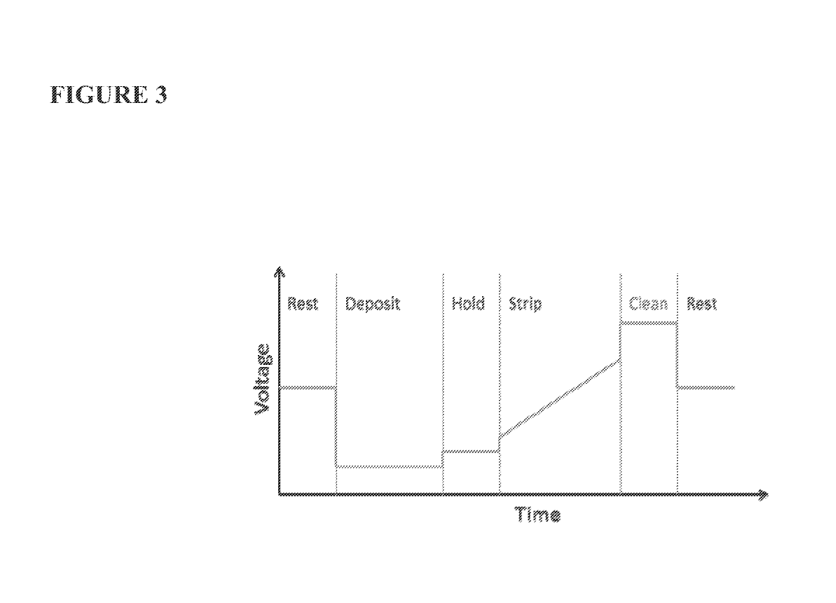

The operating process is shown in FIG. 3. The measurement operation started from a Rest stage. At the start of Rest stage, the tip of the chip was dipped into a 20 ppb standard solution of Cu, Pb and Cd. The standard solution was sucked into the flow channel by negative pressure applied at the outlet. The pressure was adjusted automatically to obtain a constant flow rate of sample, 10 ml/min for this measurement. Once the flow rate was stable, then the operation was switched to Deposit stage. A Deposit potential was applied between the working electrode and the counting electrode. After a predetermined deposit time, a Hold potential was switched on to replace Deposit potential and the negative pressure was switched off at the same time. After a predetermined Hold time had passed and the flow rate was zero, then the Strip stage was begun. The strip potential was applied between the working electrode and the counting electrode. It started from a Strip start potential and was increased to the Strip stop potential. The instrument measured the current though counting electrode during this stripping stage and metal concentrations were determined by comparing current peaks measured for the sample to a standard measurement to determine the type of metal detected. The reference electrode was used as a ground reference during measurement. The measurement was finished at the end of strip stage. The chip was then dipped into deionized water and negative pressure applied to perform a Clean operation, rinsing the flow channel with the deionized water.

After the Clean operation was completed, the chip was regenerated and used for measurement of a further sample. The measurement operations of standard solution and sample were similar, except there was no Clean operation after sample measurement. The 20 ppb Cu, Pb and Cd solution was used as sample solution. Finally the measurement result was calculated by comparison of data of standard solution measurement and sample solution measurement. This calculation was same with the calculation which was used in conventional ASV analytical methods.

Measurement Analysis

In the test experiment, the measurement of 20 ppb Cu, Pb and Cd standard solution were performed three times with three sensors.

Stock standard solutions were purchased from Merck Chemicals:

1000 mg/L Cadmium (Cd) standard solution catalog number 1197770500

1000 mg/L Copper (Cu) standard solution catalog number 1197860500

1000 mg/L Lead (Pb) standard solution catalog number 1197760500

Potassium (KNO₃) standard solution catalog number 1702300500

Nitric acid (HNO₃) catalog number 1004411000

100 ml of mixed standard solution containing 20 ppb of Cu(NO₃)₂, 20 ppb of Pb(NO₃)₂, 20 ppb of Cd(NO₃)₂, 0.1M KNO₃ and 0.1M HNO₃ was prepared by diluting the stock solutions with ultrapure water.

The experimental results obtained are shown in Table 2.

TABLE 1
Operating parameters

| | |
|---|---|
| Rest Potential (mV) | 0 |
| Flow Rate (ml/min) | 10 |
| Deposit Potential (mV) | −1000 |
| Deposit Time (s) | 30 |
| Hold Potential (mV) | −900 |
| Hold Time (s) | 10 |
| Strip Start Potential (mV) | −900 |
| Measurement Low Potential (mV) | −850 |
| Measurement High Potential (mV) | 50 |
| Strip Stop Potential (mV) | 50 |
| Strip Rate (mV/s) | 500 |
| Clean Potential (mV) | 100 |
| Clean Time (s) | 10 |

TABLE 2
Measurement result of 20 ppb Cd, Pb and Cu sample

| | Measured Concentration | Standard deviation |
|---|---|---|
| Metal | (ppb) | (ppb) |
| Cd | 21.24 | 1.33 |
| Pb | 18.88 | 1.21 |
| Cu | 20.03 | 2.12 |

What is claimed is:

1. A sensor for detecting a metal in a sample, comprising:
a microfluidic flow channel including an inlet port, an outlet port, and a detection chamber including a group of sensing electrodes including a working electrode, a counter electrode, and a reference electrode;
a flow sensor configured to measure flow in the channel;
a temperature sensor configured to measure temperature in the channel; and
an electrical connection configured to connect the sensor to a sensing device.

2. The sensor of claim 1, wherein the group of sensing electrodes includes two interdigitated electrodes and one serpentine electrode arranged between the interdigitated electrodes.

3. The sensor of claim 1, further comprising a micro-heater configured to heat a sample in the flow channel.

4. The sensor of claim 1, further comprising a pH sensor configured to measure a pH of a sample in the flow channel.

5. The sensor of claim 1, further comprising one or more sample filters.

6. The sensor of claim 1, wherein the sensor is configured to selectively detect one or more metals selected from chromium (Cr), manganese (Mn), iron (Fe), cobalt (Co), nickel (Ni), copper (Cu), zinc (Zn), arsenic (As), selenium (Se), silver (Ag), cadmium (Cd), tin (Sn), antimony (Sb), tellurium (Te), gold (Au), mercury (Hg), titanium (Ti), lead (Pb), bismuth (Bi), and a combination thereof.

7. The sensor of claim 1, wherein the flow sensor is a thermal differential sensor.

8. The sensor of claim 1, wherein the sensor is arranged on a glass substrate.

9. The sensor of claim 1, further comprising a reagent chamber configured to deliver a reagent to the flow channel.

10. The sensor of claim 9, wherein the reagent is a standard solution of copper (Cu), lead (Pb), cadmium (Cd), or a combination thereof.

11. The sensor of claim 1, wherein the electrodes are composed of a non-toxic material.

12. The sensor of claim 11, wherein the non-toxic material includes silver (Ag), gold (Au), platinum (Pt), bismuth (Bi), graphite, or glassy carbon.

13. The sensor of claim 1, wherein the electrodes are composed of mercury (Hg).

14. A system for detecting a metal in a sample comprising:
a sensing device; and
a sensor including:
a microfluidic flow channel including an inlet port, an outlet port, and a detection chamber including a group of sensing electrodes including a working electrode, a counter electrode, and a reference electrode;
a flow sensor configured to measure flow in the channel;
a temperature sensor configured to measure temperature in the channel; and
an electrical connection configured to connect the sensor to the sensing device.

15. The system of claim 14, wherein the sensing device is further connected to a computer system.

16. The system of claim 15, wherein the computer system is a smartphone.

17. The system of claim 15, wherein the computer system further comprises a computer-readable storage medium having computer-readable program code stored therein, the computer-readable program code including instructions for controlling a detection process; analysis of detection result data; and/or visualization of detection result data.

18. A method of using a sensor for detecting a metal in a sample comprising:
providing a sensor including: a microfluidic flow channel including an inlet port, an outlet port, and a detection chamber including a group of sensing electrodes including a working electrode, a counter electrode, and a reference electrode;
a flow sensor configured to measure flow in the channel;
a temperature sensor configured to measure temperature in the channel; and
an electrical connection configured to connect the sensor to a sensing device;
introducing a sample to the flow channel via the inlet port;
allowing the sample to flow to the detection chamber; and
detecting a metal in the sample using the group of sensing electrodes.

19. The method of claim 18, wherein allowing the sample to flow includes applying negative pressure to the outlet port.

20. The method of claim 19, wherein the pressure is selected to maintain a constant flow rate in the range of 0.1 ml/min to 100 ml/min.

21. The method of claim 18, wherein allowing the sample to flow includes using capillary action.

22. The method of claim 18, wherein allowing the sample to flow includes applying positive pressure to the inlet port.

23. The method of claim 18, further comprising measuring a flow rate or a flow volume of the sample in the flow channel.

24. The method of claim 23, wherein measuring the flow rate or the flow volume includes using a thermal differential sensor.

25. The method of claim 18, further comprising measuring a temperature of the sample in the flow channel.

26. The method of claim 18, further comprising applying a deposit potential between the working electrode and the counting electrode for a period of time.

27. The method of claim 26, further comprising applying a hold potential between the working electrode and the counting electrode for a period of time.

28. The method of claim 27, further comprising applying a strip potential between the working electrode and the counting electrode for a period of time.

29. The method of claim 28, further comprising measuring a current which flows through the counting electrode using a sensing device.

30. The method of claim 29, wherein a current peak is obtained from the measured current and compared with a standard measurement to determine the type of metal detected and/or the concentration of metal in the sample.

31. The method of claim 18, wherein detecting a metal using the group of sensing electrodes includes ASV or AdSV.

32. The method of claim 18, wherein the sample is a clinical sample, water sample, food sample, air sample, or soil sample.

33. The method of claim 32, wherein the food sample includes a liquid.

34. The method of claim 32, wherein the clinical sample includes stool, saliva, sputum, bronchial lavage, urine, vaginal swab, nasal swab, biopsy, tissue, tears, breath, blood, serum, plasma, cerebrospinal fluid, peritoneal fluid, pleural fluid, pericardial fluid, joint fluid, or amniotic fluid.
